(19) United States
(12) Patent Application Publication
(10) Pub. No.: US 2020/0253839 A1
(43) Pub. Date: Aug. 13, 2020

(54) COSMETIC POWDER COMPOSITIONS PROVIDING SKIN SMOOTHING AND RADIANT OPTICAL EFFECT

(71) Applicant: L'OREAL, Paris (FR)

(72) Inventor: Mariana MONTOYA, Berkeley Heights, NJ (US)

(21) Appl. No.: 16/272,229

(22) Filed: Feb. 11, 2019

(51) Int. Cl. A61K 8/02; A61K 8/19; A61K 8/81; A61K 8/58; A61K 8/73; A61Q 19/00; A61Q 1/12

(52) U.S. Cl. CPC A61K 8/022; A61K 8/19; A61K 8/8152; A61K 2800/412; A61K 8/731; A61Q 19/00; A61Q 1/12; A61K 8/585

(57) ABSTRACT

In some embodiments, a cosmetic composition includes a powder formulation, at least one additive, and at least 20% water by weight. The powder formulation includes silica silylate, methyl methacrylate crosspolymer, microcrystalline cellulose particles, and boron nitride. In some embodiments, the powder formulation includes silica silylate in an amount of about 0.05% to about 1.0%, methyl methacrylate crosspolymer in an amount of about 0.1% to about 5.0%, microcrystalline cellulose particles in an amount of about 0.1% to about 3.0%, a first portion of boron nitride in an amount of about 0.1% to about 3.0%, and a second portion of boron nitride in an amount of about 0.1% to about 5.0%, all by weight of the cosmetic composition. A method of providing a skin smoothing and radiant optical effect to skin includes applying a cosmetic composition including water, at least one additive, and a powder formulation to the skin.

### FIELD OF THE INVENTION

The invention relates to cosmetic powder formulations and cosmetic compositions and methods including such cosmetic powder formulations. More specifically, the invention relates to cosmetic powder formulations including silica silylate particles, methyl methacrylate crosspolymer particles, microcrystalline cellulose particles, and boron nitride particles and cosmetic compositions and methods including such cosmetic powder formulations.

### BACKGROUND OF THE INVENTION

Topical formulations, such as, for example, cosmetic compositions, for keratinous substrates, such as, for example, skin or hair, may include ingredients intended to provide one or more of a haze effect, a shine effect, an increased color radiance effect, and an increased radiance intensity effect to the keratinous substrate. Ingredients that provide such effects may, at higher amounts, also whiten the keratinous substrate and provide negative sensory properties to the cosmetic composition. When the keratinous substrate is skin, the whitening effect may limit the use of the cosmetic composition to lighter skin tones and the negative sensory properties may discourage use of the cosmetic composition.

There is a need for a formulation that overcomes one or more of the aforementioned drawbacks associated with products that provide one or more of a haze effect, a shine effect, an increased color radiance effect, and an increased radiance intensity effect. Such a formulation would provide a product that has pleasing sensory properties and may be used with any skin tone.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

The cosmetic compositions hereof are characterized, in various embodiments, as including powder formulations including silica silylate, methyl methacrylate crosspolymer, microcrystalline cellulose particles, and boron nitride.

In some embodiments, a cosmetic composition includes a powder formulation, at least one additive, and water. The powder formulation includes silica silylate, methyl methacrylate crosspolymer, microcrystalline cellulose particles, and boron nitride. The water is present in an amount of at least 20% by weight of the cosmetic composition.

In some embodiments, the powder formulation includes silica silylate in an amount of about 0.05% to about 1.0% by weight of the cosmetic composition, methyl methacrylate crosspolymer in an amount of about 0.1% to about 5.0% by weight of the cosmetic composition, microcrystalline cellulose particles in an amount of about 0.1% to about 3.0% by weight of the cosmetic composition, a first portion of boron nitride in an amount of about 0.1% to about 3.0% by weight of the cosmetic composition, and a second portion of boron nitride in an amount of about 0.1% to about 5.0% by weight of the cosmetic composition.

The methods hereof are characterized, in various embodiments, as providing a skin smoothing and radiant optical effect to skin by applying a cosmetic composition including a powder formulation, at least one additive, and water to the skin. The powder formulation includes silica silylate, methyl methacrylate crosspolymer, microcrystalline cellulose particles, and boron nitride.

Other features and advantages of the present invention will be apparent from the following more detailed description, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

According to the disclosure, a cosmetic composition and a method are provided that include a powder formulation.

The powder formulation provides the surprising and unexpected benefit of both a haze effect and a shine effect in a cosmetic composition for skin care without requiring silicone elastomers or silicone crosspolymers and without compromising on sensory aspects of the cosmetic composition. In some embodiments, a synergy among the distribution of particle sizes of the powder formulation may provide the benefit. In exemplary embodiments, the benefit results from a blend of powders that provide a smoothing glow effect with a minimal whitening effect, making the cosmetic composition suitable for all skin types and tones.

As used herein, whitening effect refers to the ability of a cosmetic composition to whiten a dark skin tone when applied to the skin. The whitening effect was determined based on a homogenizing power measured with a SAMBA device (Bossa Nova Vision, Los Angeles, Calif.) of a cosmetic composition on skin.

The cosmetic composition may be any water-based cosmetic composition. Appropriate types of cosmetic compositions may include, but are not limited to, skin care products or cosmetic products.

### Powder Formulation

In accordance with the disclosure, provided are cosmetic compositions that include a powder formulation. Powder formulations were developed based on screenings of the haze effect and shine effect of powder ingredients followed by testing of combinations of powder ingredients having both high haze effects and high shine effects. The powder formulation includes silica silylate, methyl methacrylate crosspolymer, microcrystalline cellulose particles, and boron nitride. In some embodiments, the powder formulation consists of silica silylate, methyl methacrylate crosspolymer, microcrystalline cellulose particles, and boron nitride. In some embodiments, the powder formulation is adjusted to improve sensory experience of the cosmetic composition without negatively affecting other desired properties of the powder formulation.

More particularly, the powder formulation may be present in the cosmetic composition at a concentration, by weight, based on the total weight of the cosmetic composition, of about 0.45% to about 17%, alternatively from about 0.45% to about 0.5%, alternatively from about 0.45% to about 1%, alternatively from about 1% to about 15%, alternatively from about 15% to about 17%, alternatively from about 16% to about 17%, or any value, range, or sub-range therebetween.

### Silica Silylate

In accordance with the disclosure, the silica silylate may be provided as a powder having a predetermined average particle size and may be a commercially-available silica silylate powder. Appropriate commercially-available silica silylate powders may include, but are not limited to, hydrophobic silica aerogels sold by Cabot Corporation (Billerica, Mass.) under the trade names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 1100®, or Enova Aerogel MT 1200®; hydrophobic silica aerogel sold by Dow Corning Corporation (Midland, Mich.) under the trade names VM-2260 or VM-2270; or combinations thereof. The particles of VM-2260 have a mean size of about 1000 microns and a specific surface area per unit of mass ranging from 600 to 800 m²/g. The particles of VM-2270 have a mean size ranging from 5-15 microns and a specific surface area per unit of mass ranging from 600 to 800 m²/g.

In some embodiments, the silica silylate powder has been formed by a sol-gel process in liquid medium followed by drying, usually by extraction of a supercritical fluid, most commonly supercritical carbon dioxide. The resulting powder particles are porous materials, typically hydrophobic silica aerogel particles. In some embodiments, the powder particles have a high average specific surface area per unit of mass (SM), such as, for example, at least 500 m²/g, alternatively at least 600, alternatively ranging from 500 to 1500 m²/g, alternatively ranging from 600 to 1200 m²/g, alternatively ranging from 600 to 800 m²/g, or any value, range, or sub-range therebetween, as determined by a Brunauer-Emmett-Teller (BET) nitrogen absorption method.

Such porous materials are conventionally described by a mean volume diameter (D[0.5]) to represent an effective average particle size. The size of the silica aerogel particles may be measured by static light scattering using a commercial granulometer such as a MasterSizer 2000 machine from Malvern Panalytical (Malvern, UK). The data are then processed on the basis of the Mie scattering theory to determine a mean volume diameter. More particularly, the silica silylate powder may have a mean volume diameter in the range of about 1 micron to about 30 microns, alternatively in the range of about 5 microns to about 25 microns, alternatively in the range of about 5 microns to about 20 microns, alternatively in the range of about 5 microns to about 15 microns, or any value, range, or sub-range therebetween.

More particularly, the silica silylate powder may be present in the powder formulation at a weight ratio, based on the total weight of the powder formulation, of about 0.05 to about 0.1, alternatively from about 0.05 to about 0.06, alternatively from about 0.06 to about 0.08, alternatively from about 0.09 to about 0.1, alternatively about 0.06, alternatively about 0.07, alternatively about 0.08, alternatively about 0.09, or any value, range, or sub-range therebetween.

More particularly, the silica silylate powder may be present in the cosmetic composition at a concentration, by weight, based on the total weight of the cosmetic composition, of about 0.05% to about 1%, alternatively from about 0.05% to about 0.1%, alternatively from about 0.05% to about 0.2%, alternatively from about 0.2% to about 0.5%, alternatively from about 0.2% to about 0.8%, alternatively from about 0.5% to about 0.8%, alternatively from about 0.8% to about 1%, or any value, range, or sub-range therebetween. In exemplary embodiments, the amount of the silica silylate powder does not exceed 0.5% by weight of the cosmetic composition due to negative impacts on texture occurring at higher concentrations of the silica silylate powder.

### Methyl Methacrylate Crosspolymer

In accordance with the disclosure, the methyl methacrylate crosspolymer may be provided as a powder having a predetermined average particle size and may be a commercially-available methyl methacrylate crosspolymer powder. More particularly, the methyl methacrylate crosspolymer powder may have an average particle size in the range of about 8 microns to about 12 microns, alternatively in the range of about 9 microns to about 11 microns, alternatively in the range of about 9.5 microns to about 10.5 microns, alternatively about 10 microns, or any value, range, or sub-range therebetween.

More particularly, the methyl methacrylate crosspolymer powder may be present in the powder formulation at a weight ratio, based on the total weight of the powder formulation, of about 0.2 to about 0.6, alternatively from about 0.2 to about 0.3, alternatively from about 0.3 to about 0.5, alternatively from about 0.5 to about 0.6, alternatively about 0.2, alternatively about 0.3, alternatively about 0.4, alternatively about 0.5, alternatively about 0.6, or any value, range, or sub-range therebetween.

More particularly, the methyl methacrylate crosspolymer powder may be present in the cosmetic composition at a concentration, by weight, based on the total weight of the cosmetic composition, of about 0.1% to about 5%, alternatively from about 0.1% to about 0.5%, alternatively from about 0.5% to about 1%, alternatively from about 1% to about 2%, alternatively from about 2% to about 4%, alternatively from about 4% to about 5%, or any value, range, or sub-range therebetween. In exemplary embodiments, the amount of the methyl methacrylate crosspolymer powder does not exceed 4% by weight of the cosmetic composition to limit a whitening effect occurring at higher concentrations of the methyl methacrylate crosspolymer powder in combination with other powders of the powder formulation.

### Microcrystalline Cellulose Particles

In accordance with the disclosure, the powder of microcrystalline cellulose particles may be provided as solid particles of cellulose in a powder form having a predetermined average particle size and may be a commercially-available material. Appropriate commercially-available cellulose particles may include, but are not limited to, spherical cellulose beads such as those sold by Daito Kasei Kogyo Co., Ltd. (Osaka, Japan) under the trade name Cellulobeads USF, those sold by FMC Corporation (Philadelphia, Pa.) under the trade name Avicel® PH 105 NF, and those sold by J. Rettenmaier & Söhne GmbH & Co. (Rosenberg, Germany) under the trade name Vivapur® CS 9FM. More particularly, the microcrystalline cellulose particles may have an average particle size in the range of about 2 microns to about 6 microns, alternatively in the range of about 3 microns to about 5 microns, alternatively in the range of about 3.5 microns to about 4.5 microns, alternatively about 4 microns, or any value, range, or sub-range therebetween.

More particularly, the microcrystalline cellulose particles may be present in the powder formulation at a weight ratio, based on the total weight of the powder formulation, of about 0.05 to about 0.4, alternatively from about 0.05 to about 0.1, alternatively from about 0.1 to about 0.2, alternatively from about 0.2 to about 0.3, alternatively from about 0.3 to about 0.4, alternatively about 0.05, alternatively about 0.1, alternatively about 0.2, alternatively about 0.3, alternatively about 0.4, or any value, range, or sub-range therebetween.

More particularly, the microcrystalline cellulose particles may be present in the cosmetic composition at a concentration, by weight, based on the total weight of the cosmetic composition, of about 0.1% to about 3%, alternatively from about 0.1% to about 0.5%, alternatively from about 0.5% to about 1%, alternatively from about 1% to about 2%, alternatively from about 2% to about 3%, alternatively about 1%, alternatively about 2%, alternatively about 3%, or any value, range, or sub-range therebetween. In exemplary embodiments, the amount of the microcrystalline cellulose particles does not exceed 1.5% by weight of the cosmetic composition due to negative impacts on skin formula sensory occurring at higher concentrations of the microcrystalline cellulose particles.

### Boron Nitride

In accordance with the disclosure, the boron nitride may be provided as one or more powders having a predetermined average particle size and may be a commercially-available boron nitride powder. Appropriate commercially-available boron nitride powders may include, but are not limited to, those sold by Mizushima Ferroalloy Co., Ltd. (Kurashiki, Japan) under the trade name SHP series having a mean or average particle size ranging from about 3 microns to about 18 microns; those sold by Saint-Gobain Manufacturing Company (Courbevoie, France) under the trade name PUHP3008 having a mean particle size of about 6 microns and under the trade name PUHP1030L having a mean particle size of about 3 microns; those sold by Momentive Performance Materials, Inc. (Waterford, N.Y.) under the trade name Softouch® BN CC6058 having a mean particle size of about 5 microns to about 15 microns; or combinations thereof.

The boron nitride powder may include a first boron nitride powder having a first predetermined average particle size and a second boron nitride powder having a second predetermined average particle size. More particularly, the first boron nitride powder may have a first average particle size in the range of about 10 microns to about 20 microns, alternatively in the range of about 12 microns to about 18 microns, alternatively in the range of about 14 microns to about 16 microns, alternatively about 15 microns, or any value, range, or sub-range therebetween, and the second boron nitride may have a second average particle size in the range of about 40 microns to about 55 microns, alternatively in the range of about 40 microns to about 50 microns, alternatively in the range of about 46 microns to about 48 microns, alternatively about 47 microns, or any value, range, or sub-range therebetween.

In some embodiments, the weight ratio of the first boron nitride powder to the second boron nitride powder is in the range of about 2:1 to about 4:1, alternatively about 2:1 to about 3:1, alternatively about 3:1 to about 4:1, alternatively about 2:1, alternatively about 3:1, alternatively about 4:1, or any value, range, or sub-range therebetween. In some embodiments, one boron nitride powder gives a good soft focus property and the other boron nitride powder gives a good sparkling property, but their combination yields a good luminosity not provided by the boron nitride powders individually and a synergistic glow.

More particularly, the first boron nitride powder may be present in the powder formulation at a weight ratio, based on the total weight of the powder formulation, of about 0.2 to about 0.5, alternatively from about 0.2 to about 0.3, alternatively from about 0.3 to about 0.4, alternatively from about 0.4 to about 0.5, alternatively about 0.2, alternatively about 0.3, alternatively about 0.4, alternatively about 0.5, or any value, range, or sub-range therebetween.

More particularly, the first boron nitride powder may be present in the cosmetic composition at a concentration, by weight, based on the total weight of the cosmetic composition, of about 0.1% to about 3%, alternatively from about 0.1% to about 0.5%, alternatively from about 0.5% to about 1%, alternatively from about 1% to about 2%, alternatively from about 2% to about 3%, alternatively from about 2.5% to about 3%, alternatively about 1%, alternatively about 2%, alternatively about 3%, or any value, range, or sub-range therebetween. In exemplary embodiments, the amount of the first boron nitride powder does not exceed 2% by weight of the cosmetic composition to limit a whitening effect occurring at higher concentrations of the first boron nitride powder.

More particularly, the second boron nitride powder may be present in the powder formulation at a weight ratio, based on the total weight of the powder formulation, of about 0.1 to about 0.7, alternatively from about 0.1 to about 0.2, alternatively from about 0.2 to about 0.3, alternatively from about 0.3 to about 0.5, alternatively from about 0.5 to about 0.6, alternatively from about 0.6 to about 0.7, alternatively about 0.2, alternatively about 0.3, alternatively about 0.4, alternatively about 0.5, alternatively about 0.6, or any value, range, or sub-range therebetween. In exemplary embodiments, the second boron nitride powder is provided in the powder formulation in an amount to have a low impact on haze in RADAR but to contribute more to shine.

More particularly, the second boron nitride powder may be present in the cosmetic composition at a concentration, by weight, based on the total weight of the cosmetic composition, of about 0.1% to about 5%, alternatively from about 0.1% to about 0.5%, alternatively from about 0.5% to about 1%, alternatively from about 1% to about 2%, alternatively from about 2% to about 4%, alternatively from about 4% to about 5%, or any value, range, or sub-range therebetween.

### Additives

In accordance with the disclosure, the composition further includes at least one additive. The at least one additive may be selected to provide a cosmetic composition of a predetermined type having at least one predetermined property. The total amount of the at least one additive may be any appropriate amount, such as, for example, about 60% or less, alternatively from about 40% to about 60%, alternatively from about 45% to about 55%, alternatively about 45%, alternatively about 50%, alternatively about 55%, or any suitable value, range, or sub-range thereof, by weight, based on the weight of the composition.

The at least one additive may include, but is not limited to, at least one active, at least one binder, at least one fatty compound, at least one pearl pigment, at least one polymer, at least one preservative, at least one silicone, at least one surfactant, at least one non-water solvent, at least one adjuvant, or combinations thereof.

The cosmetic composition may include any number of appropriate actives as additives. Appropriate actives for the cosmetic composition may include, but are not limited to, adenosine, capryloyl salicylic acid, glycolic acid, sodium hyaluronate, sodium hydroxide, trisodium ethylene diamine disuccinate, or combinations thereof. The total amount of the actives may be any appropriate amount, such as, for example, about 15% or less, alternatively from about 5% to about 15%, alternatively from about 10% to about 3%, by weight, based on the weight of the composition.

The cosmetic composition may include any number of appropriate solid or liquid binders as additives. An appropriate solid binder may include, but is not limited to, magnesium stearate. An appropriate liquid binder may include, but is not limited to, a polyethylene glycol (PEG) dimethicone.

The cosmetic composition may include any number of appropriate fatty compounds as additives. Appropriate fatty compounds for the cosmetic composition may include, but are not limited to, polyglyceryl-3 methylglucose distearate. The total amount of the fatty compounds may be any appropriate amount, such as, for example, about 5% or less, alternatively about 3% or less, alternatively from about 1% to about 3%, by weight, based on the weight of the composition.

The cosmetic composition may include any number of appropriate pearl pigments as additives. Appropriate pearl pigments for the cosmetic composition may include, but are not limited to, composite particles including a substrate formed from mica, synthetic fluorphlogopite coated with at least one mineral pigment and at least one pearl pigment, chosen from composite particles including at least one support chosen from mica, synthetic fluorphlogopite or calcium sodium borosilicate, and completely or partially coated with one or more layers of metal oxides, in particular chosen from titanium dioxide, iron oxide, tin oxide, or combinations thereof. The total amount of the pearl pigments may be any appropriate amount, such as, for example, about 2% or less, alternatively from about 1% to about 2%, by weight, based on the weight of the composition.

The cosmetic composition may include any number of appropriate polymers as additives. Appropriate polymers for the cosmetic composition may include, but are not limited to, ammonium polyacryloyldimenthyl taurate, hydroxyethyl acrylate/sodium acryloyldimethyl taurate copolymer, polyacrylamide (and) C13-C14 isoparaffin (and) Laureth-7, or combinations thereof. The total amount of the polymers may be any appropriate amount, such as, for example, about 5% or less, alternatively from about 1% to about 3%, alternatively from about 2% to about 2.5%, by weight, based on the weight of the composition.

The cosmetic composition may include any number of appropriate preservatives as additives. Appropriate preservatives for the cosmetic composition may include, but are not limited to, chlorphenesin, phenoxyethanol, or combinations thereof. The total amount of the preservatives may be any appropriate amount, such as, for example, about 1% or less, alternatively from about 0.3% to about 0.7%, by weight, based on the weight of the composition.

The cosmetic composition may include any number of appropriate silicones as additives. Appropriate silicones for the cosmetic composition may include, but are not limited to, cyclohexasiloxane, dimethicone, dimethicone and dimethicone/vinyl dimethicone crosspolymer, or combinations thereof. The total amount of the silicones may be any appropriate amount, such as, for example, about 25% or less, alternatively from about 20% to about 25%, alternatively about 20% or less, alternatively about 15% or less, alternatively about 10% or less, alternatively about 5% or less, by weight, based on the weight of the composition.

The cosmetic composition may include any number of appropriate non-water solvents as additives. Appropriate non-water solvents for the cosmetic composition may include, but are not limited to, denatured alcohol, caprylyl glycol, glycerin, or combinations thereof. The total amount of the non-water solvents may be any appropriate amount, such as, for example, about 20% or less, alternatively from about 10% to about 20%, alternatively from about 15% to about 20%, by weight, based on the weight of the composition.

The cosmetic composition may include any number of appropriate surfactants as additives. Appropriate surfactants for the cosmetic composition may include, but are not limited to, glyceryl stearate, myristic acid, palmitic acid, stearic acid, PEG-100 stearate, steareth-100, or combinations thereof. The total amount of the surfactants may be any appropriate amount, such as, for example, about 8% or less, alternatively from about 4% to about 8%, by weight, based on the weight of the composition.

The cosmetic composition may include any number of other appropriate additives or adjuvants. Representative additives and adjuvants include, for example, water-soluble or water-miscible solvents or co-solvents, dispersion enhancing agents, moisturizers, colorants, fillers, antioxidants (e.g., ethylenediaminetetraacetic acid (EDTA), butylated hydroxytoluene (BHT), and tocopherol), essential oils, fragrances, dyes, neutralizing or pH-adjusting agents (e.g., citric acid, triethylamine (TEA), and sodium hydroxide), conditioning or softening agents (e.g., panthenol and allantoinin) and extracts, such as botanical extracts. Additives and adjuvants may be present in the compositions in amounts generally ranging from about 0.01% to about 10%, by weight. Examples of cosmetic active agents or dermatological active agents include sunscreen agents (e.g., inorganic sunscreen agents, such as titanium dioxide and zinc oxide and organic sunscreen agents, such as octocrylene, ethylhexyl methoxycinnamate, and avobenzone), free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents.

### Water

In accordance with the disclosure, the cosmetic composition is a water-based composition.

In accordance with the various embodiments, water is present in the composition at a concentration, by weight, based on the total weight of the composition, of about 20% or greater, alternatively about 37% or greater, alternatively about 40% or greater, alternatively about 70% or less, alternatively from about 20% to about 70%, alternatively from about 20% to about 50%, alternatively from about 35% to about 50%, alternatively from about 35% to about 40%, alternatively from about 40% to about 45%, alternatively from about 45% to about 50%, alternatively from about 40% to about 60%, alternatively from about 50% to about 70%, alternatively from about 50% to about 60%, alternatively about 35%, 36%, 37%, 38%, 39%, 40%, 41%, 42%, 43%, 44%, 45%, 46%, 47%, 48%, 49%, 50%, or any suitable value, range, or sub-range thereof.

In other embodiments, such as, for example, in a primer, the cosmetic composition is anhydrous or substantially anhydrous. In such embodiments, the quantum satis (QS) may be silicone, for example.

### Raw Materials

Compositions as described in the representative embodiments according to the disclosure, and compositions as exemplified herein include raw materials selected from commercially available materials.

### Examples

The invention is further described in the context of the following examples, which are presented by way of illustration, not of limitation. The inventive compositions may be used as a skin care or cosmetic product, such as, for example, a serum, a lotion, a liquid, a cream, a gel, an emulsion, a primer, or a BB cream.

### Compositions

Two commercially-available comparative compositions were tested. TABLE 1 lists the ingredients and their amounts in weight percentage in Comparative Example 1 and Comparative Example 2. Comparative Example 1 is Haze Effect Peach Effect (Pro-Xylane) and Comparative Example 2 is Maybelline Baby Skin Instant Pore Eraser. The Comparative Examples lack the powder formulation.

TABLE 1
Comparative Compositions

| | | COMPAR- | COMPAR- |
|---|---|---|---|
| | | ATIVE | ATIVE |
| TYPE | INGREDIENT | 1 | 2 |
| ACTIVE | CITRIC ACID | 0.05 | |
| COMPOUND | | | |
| ACTIVE | DISODIUM EDTA | 0.1 | |
| COMPOUND | | | |
| ACTIVE | HYDROXYPROPYL | 9 | |
| COMPOUND | TETRAHYDROPYRANTRIOL | | |
| ACTIVE | SODIUM CHLORIDE | 1 | |
| COMPOUND | | | |
| ACTIVE | SODIUM CITRATE | 0.2 | |
| COMPOUND | | | |
| DYE/PIGMENT | IRON OXIDES | | 0.000564 |
| DYE/PIGMENT | RED 30 | | 0.000579 |
| DYE/PIGMENT | RED 4 | 0.00018 | |
| DYE/PIGMENT | YELLOW 6 | 0.00038 | |
| FATTY | ISOPROPYL ISOSTEARATE | 1 | |
| COMPOUND | | | |
| FATTY | PENTAERYTHRITYL | | 0.002197 |
| COMPOUND | TETRAISOSTEARATE | | |
| FATTY | STEARYL HEPTANOATE | | 10 |
| COMPOUND | | | |
| FILLER | SILICA SILYLATE | 0.3 | 0.25 |
| POLYMER | HDI/TRIMETHYLOL | 4 | |
| | HEXYLLACTONE | | |
| | CROSSPOLYMER | | |
| POLYMERIC | METHYL METHACRYLATE | 4 | |
| FILLER | CROSSPOLYMER (and) | | |
| | MINERAL OIL | | |
| PRESERVATIVE | CHLORPHENESIN | 0.3 | |
| PRESERVATIVE | PHENOXYETHANOL | 0.5 | |
| SILICONE | DIMETHICONE | 8 | 10.89666 |
| SILICONE | DIMETHICONE (and) | | 78.5 |
| | DIMETHICONE | | |
| | CROSSPOLYMER | | |
| SILICONE | DIMETHICONE (and) | 7 | |
| | DIMETHICONE/PEG-10/15 | | |
| | CROSSPOLYMER | | |
| SILICONE | DIMETHICONE (and) | 10 | |
| | DIMETHICONE/VINYL | | |
| | DIMETHICONE | | |
| | CROSSPOLYMER | | |
| SILICONE | PEG-10 DIMETHICONE | 0.07 | |
| SOLVENT | CAPRYLYL GLYCOL | 0.3 | 0.3 |
| SOLVENT | GLYCERIN | 15 | |
| SOLVENT | PROPANEDIOL | 3 | |
| SOLVENT | WATER | 36.17944 | |
| VEGETAL | PROPYLENE GLYCOL (and) | | 0.05 |
| EXTRACT | PRUNUS CERASUS (BITTER | | |
| | CHERRY) EXTRACT | | |

Five inventive compositions were formed that included a powder formulation including silica silylate, methyl methacrylate crosspolymer, microcrystalline cellulose particles, and boron nitride. TABLE 2 lists the ingredients and their amounts in weight percentage in Inventive Example 1 and Inventive Example 2. TABLE 3 lists the ingredients and their amounts in weight percentage in Inventive Example 3, Inventive Example 4, and Inventive Example 5. The compositions of Inventive Examples 1, 2, 3, 4, and 5 include the powder formulation.

TABLE 2
Inventive Compositions 1-2

| | INVENTIVE | INVENTIVE |
|---|---|---|
| INGREDIENT | 1 | 2 |
| ADENOSINE | 0.1 | 0.1 |
| ALCOHOL DENATURED | 3 | |
| CAPRYLOYL SALICYLIC ACID | 0.3 | 0.3 |
| GLYCERIN | 15 | 15 |
| GLYCOLIC ACID | 8.4 | 8.4 |
| PHENOXYETHANOL | 0.5 | 0.5 |
| SODIUM HYALURONATE | 0.2 | 0.2 |
| SODIUM HYDROXIDE | 2.2 | 2.2 |
| TRISODIUM ETHYLENEDIAMINE DISUCCINATE | 0.1 | 0.1 |
| WATER | 48.45 | 37.7 |
| METHYL METHACRYLATE CROSSPOLYMER | 2 | 2 |
| BUTYROSPERMUM PARKII (SHEA) BUTTER | 2 | |
| CAPRYLIC/CAPRIC TRIGLYCERIDE | 6 | |
| CYCLOHEXASILOXANE | | 4 |
| DIMETHICONE | | 8 |
| DIMETHICONE (and) DIMETHICONE/VINYL | | 10 |
| DIMETHICONE CROSSPOLYMER | | |
| OCTYLDODECANOL | 0.5 | |
| PENTAERYTHRITYL TETRAETHYLHEXANOATE | 1 | |
| AMMONIUM POLYACRYLOYLDIMETHYL | 1.5 | 1 |
| TAURATE | | |
| HYDROXYETHYL ACRYLATE/SODIUM | | 0.75 |
| ACRYLOYLDIMETHYL TAURATE COPOLYMER | | |
| POLYACRYLAMIDE (and) C13-14 ISOPARAFFIN | 0.6 | 0.6 |
| (and) LAURETH-7 | | |
| CAPRYLYL GLYCOL | 0.3 | 0.3 |
| POLYGLYCERYL-3 METHYLGLUCOSE | 1.5 | 2.5 |
| DISTEARATE | | |
| BORON NITRIDE (47 microns) | 2 | 2 |
| BORON NITRIDE (15 microns) | 0.5 | 0.5 |
| MICROCRYSTALLINE CELLULOSE | 1.5 | 1.5 |
| (4 microns or 9 microns) | | |
| SILICA SILYLATE | 0.5 | 0.5 |
| MICA (and) IRON OXIDES (and) TITANIUM DIOXIDE | 0.3 | 0.3 |
| MICA (and) TITANIUM DIOXIDE (and) IRON OXIDES | 0.5 | 0.5 |
| SYNTHETIC FLUORPHLOGOPITE | 0.4 | 0.4 |
| SYNTHETIC FLUORPHLOGOPITE (and) TITANIUM | 0.65 | 0.65 |
| DIOXIDE (and) TIN OXIDE | | |

TABLE 3
Inventive Compositions 3-5

| | INVENTIVE | INVENTIVE | INVENTIVE |
|---|---|---|---|
| INGREDIENT | 3 | 4 | 5 |
| ADENOSINE | 0.1 | 0.1 | 0.1 |
| ALCOHOL DENATURED | 3 | 3 | |
| CAPRYLOYL SALICYLIC ACID | 0.3 | 0.3 | 0.3 |
| GLYCERIN | 15 | 15 | 15 |
| GLYCOLIC ACID | 8.4 | 8.4 | 8.4 |
| PHENOXYETHANOL | 0.5 | 0.5 | 0.5 |
| SODIUM HYALURONATE | 0.2 | 0.2 | 0.2 |
| SODIUM HYDROXIDE | 2.2 | 2.2 | 2.2 |
| TRISODIUM ETHYLENEDIAMINE | 0.1 | 0.1 | 0.1 |
| DISUCCINATE | | | |
| WATER | 46.7 | 48.55 | 43.7 |
| METHYL METHACRYLATE | 3 | 3 | 3 |
| CROSSPOLYMER | | | |
| BUTYROSPERMUM PARKII (SHEA) | 2 | 2 | |
| BUTTER | | | |
| CAPRYLIC/CAPRIC TRIGLYCERIDE | 7 | 7 | 3 |
| CAPRYLYL METHICONE | | | 2 |
| DIMETHICONE (and) | | | 10 |
| DIMETHICONE/VINYL DIMETHICONE | | | |
| CROSSPOLYMER | | | |
| OCTYLDODECANOL | 0.5 | 0.5 | |
| PENTAERYTHRITYL | 1 | 1 | |
| TETRAETHYLHEXANOATE | | | |
| AMMONIUM | 1.75 | 1.75 | 1 |
| POLYACRYLOYLDIMETHYL TAURATE | | | |
| HYDROXYETHYL ACRYLATE/SODIUM | | | 1.25 |
| ACRYLOYLDIMETHYL TAURATE | | | |
| COPOLYMER | | | |
| POLYACRYLAMIDE (and) C13-14 | 0.6 | 0.6 | 0.6 |
| ISOPARAFFIN (and) LAURETH-7 | | | |
| CAPRYLYL GLYCOL | 0.3 | 0.3 | 0.3 |
| POLYGLYCERYL-3 METHYLGLUCOSE | 1.5 | 1.5 | 2.5 |
| DISTEARATE | | | |
| BORON NITRIDE (47 microns) | 2 | 2 | 2 |
| BORON NITRIDE (15 microns) | 1 | 1 | 1 |
| MICROCRYSTALLINE CELLULOSE | 0.5 | 0.5 | 0.5 |
| PARTICLES | | | |
| SILICA SILYLATE | 0.5 | 0.5 | 0.5 |
| MICA (and) IRON OXIDES (and) | 0.3 | | 0.3 |
| TITANIUM DIOXIDE | | | |
| MICA (and) TITANIUM DIOXIDE (and) | 0.5 | | 0.5 |
| IRON OXIDES | | | |
| SYNTHETIC FLUORPHLOGOPITE | 0.4 | | 0.4 |
| SYNTHETIC FLUORPHLOGOPITE (and) | 0.65 | | 0.65 |
| TITANIUM DIOXIDE (and) TIN OXIDE | | | |

### Methods of Preparation

To form the Inventive Examples, the water phase ingredients, including the water, emollients, preservatives, glycolic acid, adenosine, and capryloyl salicylic acid, were combined and heated to about 80° C. The methyl methacrylate crosspolymer may be introduced at this time to avoid aeration when scaling up. This homogenized water phase was then cooled to 65° C., where it emulsified with the oil phase. As the water phase cooled, the thickening system was to turn system into a gel. The oil and silicone phase may be heated separately and added all at once, or if using a more efficient all-in-one process, the emulsifier may be added to the water phase and homogenized in followed by the remaining oils and silicones. Once emulsified, the emulsion was cooled to 30° C. Once the emulsion was cooled, the boron nitrides, cellulose, silica silylate, and methyl methacrylate crosspolymer, if it had not yet been added, were added. It was important not to homogenize at this point, as this can impact the particle size of these ingredients. Last, the pearls were swept into the batch. Sweeping continued until homogenous.

### Testing Results

Comparative Example 1, Comparative Example 2, Inventive Example 3, Inventive Example 4, and Inventive Example 5 were tested on bare skin for shine, radiance, and color change relative to the bare skin. Measurements were taken 15 minutes, one hour, and six hours after applying the Example compositions.

A SAMBA shine analysis compared the shine of Comparative Example 1, Comparative Example 2, Inventive Example 3, Inventive Example 4, and Inventive Example 5 applied to bare skin relative to the shine of the bare skin without application of the Example compositions. TABLE 4 shows an increase in shine value from Inventive Example 4 and significant increases in shine value from Inventive Example 3 and Inventive Example 5 at all three test times, indicating an early and long-term shine effect. The shine value decreased for the two Comparative Examples. The amount of whitening was also measured as a homogenizing power by the SAMBA.

TABLE 4
SAMBA Shine Analysis

| | Baseline | 15 Minutes | 1 Hour | 6 Hours |
|---|---|---|---|---|
| Bare Skin | 38.43 ± 5.51 | 39.68 ± 7.30 | 40.59 ± 8.34 | 41.05 ± 8.87 |
| Comparative | 39.40 ± 6.26 | 36.42 ± 5.60 | 35.11 ± 4.84 | 35.98 ± 4.88 |
| Example 1 | | | | |
| Comparative | 38.78 ± 5.23 | 33.06 ± 3.29 | 34.18 ± 3.90 | 37.12 ± 5.01 |
| Example 2 | | | | |
| Inventive | 38.90 ± 5.16 | 48.99 ± 8.12 | 50.11 ± 8.88 | 49.16 ± 7.98 |
| Example 3 | | | | |
| Inventive | 39.50 ± 5.25 | 42.39 ± 8.12 | 42.98 ± 4.64 | 43.25 ± 7.45 |
| Example 4 | | | | |
| Inventive | 40.73 ± 5.60 | 49.42 ± 5.99 | 49.97 ± 6.13 | 48.38 ± 7.11 |
| Example 5 | | | | |

A radiance color analysis tested the immediate and long-term color radiance effects of application of Comparative Example 1, Comparative Example 2, Inventive Example 3, Inventive Example 4, and Inventive Example 5 to bare skin relative to the color radiance of the bare skin without application of the Example compositions. TABLE 5 shows a significant increase in color radiance from all Inventive Examples at all three test times, indicating an early and long-term color radiance effect. Inventive Examples 3 and 5 again performed better than Inventive Example 4. The Comparative Examples provided no measurable change in color radiance.

TABLE 5
Radiance Color Analysis

| | Baseline | 15 Minutes | 1 Hour | 6 Hours |
|---|---|---|---|---|
| Bare Skin | 4.66 ± 0.67 | 4.57 ± 1.19 | 4.89 ± 1.01 | 4.92 ± 1.16 |
| Comparative | 4.67 ± 0.62 | 4.91 ± 0.45 | 4.79 ± 0.47 | 4.62 ± 0.78 |
| Example 1 | | | | |
| Comparative | 4.79 ± 0.51 | 4.90 ± 0.59 | 5.00 ± 0.75 | 4.85 ± 0.88 |
| Example 2 | | | | |
| Inventive | 4.73 ± 0.43 | 7.04 ± 1.34 | 7.14 ± 1.04 | 6.46 ± 0.37 |
| Example 3 | | | | |
| Inventive | 4.78 ± 0.51 | 5.93 ± 0.80 | 5.95 ± 0.63 | 5.54 ± 0.65 |
| Example 4 | | | | |
| Inventive | 4.76 ± 0.73 | 6.42 ± 0.69 | 6.64 ± 0.83 | 6.49 ± 0.55 |
| Example 5 | | | | |

A radiance intensity analysis tested the immediate and long-term radiance intensity effects of application of Comparative Example 1, Comparative Example 2, Inventive Example 3, Inventive Example 4, and Inventive Example 5 to bare skin relative to radiance intensity of the bare skin without application of the Example compositions. TABLE 6 shows a significant increase in radiance intensity from all Inventive Examples at all three test times, indicating an early and long-term radiance intensity effect. Again, Inventive Examples 3 and 5 performed better than Inventive Example 4. The Comparative Examples provided no measurable change in radiance intensity.

TABLE 6
Radiance Intensity Analysis

| | Baseline | 15 Minutes | 1 Hour | 6 Hours |
|---|---|---|---|---|
| Bare Skin | 5.15 ± 0.22 | 5.43 ± 0.84 | 5.52 ± 0.52 | 5.70 ± 0.53 |
| Comparative | 5.20 ± 0.25 | 5.14 ± 0.57 | 5.01 ± 0.61 | 5.01 ± 0.59 |
| Example 1 | | | | |
| Comparative | 5.27 ± 0.20 | 5.41 ± 0.41 | 5.55 ± 0.61 | 5.37 ± 0.50 |
| Example 2 | | | | |
| Inventive | 5.35 ± 0.29 | 7.01 ± 0.62 | 7.01 ± 0.60 | 6.65 ± 0.48 |
| Example 3 | | | | |
| Inventive | 5.44 ± 0.32 | 6.08 ± 0.67 | 6.18 ± 0.52 | 5.93 ± 0.47 |
| Example 4 | | | | |
| Inventive | 5.30 ± 0.34 | 6.66 ± 0.78 | 6.85 ± 0.82 | 6.73 ± 0.69 |
| Example 5 | | | | |

A chromasphere analysis tested the immediate color change upon application of Comparative Example 1, Comparative Example 2, Inventive Example 3, Inventive Example 4, and Inventive Example 5 to bare skin. TABLE 7 shows a minimum visible color shift (Delta E94 less than 2.0) from all tests at all three test times.

TABLE 7
Chromasphere Analysis

| | Baseline | 15 Minutes | 1 Hour | 6 Hours |
|---|---|---|---|---|
| Bare Skin | 0 | 0.80 ± 0.57 | 0.82 ± 0.46 | 1.42 ± 0.69 |
| Comparative | 0 | 1.01 ± 0.75 | 0.95 ± 0.62 | 1.90 ± 1.20 |
| Example 1 | | | | |
| Comparative | 0 | 1.06 ± 0.64 | 0.88 ± 0.52 | 1.50 ± 1.07 |
| Example 2 | | | | |
| Inventive | 0 | 1.56 ± 1.31 | 1.14 ± 0.98 | 1.92 ± 0.81 |
| Example 3 | | | | |
| Inventive | 0 | 1.18 ± 0.49 | 1.49 ± 0.91 | 1.49 ± 0.75 |
| Example 4 | | | | |
| Inventive | 0 | 1.93 ± 1.59 | 1.52 ± 0.97 | 1.65 ± 1.23 |
| Example 5 | | | | |

While the two Comparative Examples were able to provide a similar desirable blurring efficacy to the five Inventive Examples, the Comparative Examples were not able to deliver the increase in shine provided by the Inventive Examples.

The articles “a” and “an”, as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more”, as used herein, means at least one, and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about”, meaning within 10% of the indicated number (e.g. “about 10%” means 9% to 11% and “about 2%” means 1.8% to 2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A cosmetic composition, comprising:
a powder formulation comprising:
silica silylate;
methyl methacrylate crosspolymer;
microcrystalline cellulose particles; and
boron nitride;
at least one additive; and
water in an amount of at least 20% by weight of the cosmetic composition.

2. The cosmetic composition of claim 1, wherein the cosmetic composition, when applied to a keratinous surface, provides the keratinous surface with an optical haze, a shine, and a radiance and provides no more than a minimum whitening effect.

3. The cosmetic composition of claim 1, wherein the silica silylate has a mean volume diameter of about 1 micron to about 30 microns.

4. The cosmetic composition of claim 1, wherein the silica silylate is present in an amount of about 0.05% to about 1.0% by weight of the cosmetic composition.

5. The cosmetic composition of claim 1, wherein the methyl methacrylate crosspolymer has an average particle size of about 10 microns.

6. The cosmetic composition of claim 1, wherein the methyl methacrylate crosspolymer is present in an amount of about 0.1% to about 5.0% by weight of the cosmetic composition.

7. The cosmetic composition of claim 1, wherein the microcrystalline cellulose particles have an average particle size of about 4 microns.

8. The cosmetic composition of claim 1, wherein the microcrystalline cellulose particles are present in an amount of about 0.1% to about 3.0% by weight of the cosmetic composition.

9. The cosmetic composition of claim 1, wherein the boron nitride consists of a first portion having an average particle size of about 15 microns and a second portion having an average particle size of about 47 microns.

10. The cosmetic composition of claim 9, wherein the first portion of boron nitride is present in an amount of about 0.1% to about 3.0% by weight of the cosmetic composition.

11. The cosmetic composition of claim 9, wherein the second portion of boron nitride is present in an amount of about 0.1% to about 5.0% by weight of the cosmetic composition.

12. The cosmetic composition of claim 1, wherein the powder formulation is about 0.45% to about 17% by weight of the cosmetic composition.

13. The cosmetic composition of claim 1, wherein the water is present in an amount of at least 37% by weight of the cosmetic composition.

14. A cosmetic composition, comprising:
a powder formulation comprising:
silica silylate in an amount of about 0.05% to about 1.0% by weight of the cosmetic composition;
methyl methacrylate crosspolymer in an amount of about 0.1% to about 5.0% by weight of the cosmetic composition;
microcrystalline cellulose particles in an amount of about 0.1% to about 3.0% by weight of the cosmetic composition;
a first portion of boron nitride in an amount of about 0.1% to about 3.0% by weight of the cosmetic composition; and
a second portion of boron nitride in an amount of about 0.1% to about 5.0% by weight of the cosmetic composition; and
at least one additive; and
water in an amount of at least 20% by weight of the cosmetic composition.

15. The cosmetic composition of claim 14, wherein the silica silylate has a mean volume diameter of about 1 micron to about 30 microns, the methyl methacrylate crosspolymer has an average particle size of about 10 microns, the microcrystalline cellulose particles have an average particle size of about 4 microns, the first portion of boron nitride has an average particle size of about 15 microns, and the second portion of boron nitride has an average particle size of about 47 microns.

16. The cosmetic composition of claim 14, wherein the water is present in an amount of at least 37% by weight of the cosmetic composition.

17. A method of providing a skin smoothing and radiant optical effect to skin, the method comprising:
applying a cosmetic composition comprising water in an amount of at least 20% by weight of the cosmetic composition, at least one additive, and a powder formulation comprising silica silylate, methyl methacrylate crosspolymer, microcrystalline cellulose particles, and boron nitride to the skin.

18. The method of claim 17 further comprising combining the water, the at least one additive, and the powder formulation to form the cosmetic composition.

19. The method of claim 17, wherein the silica silylate has a mean volume diameter of about 1 micron to about 30 microns, the methyl methacrylate crosspolymer has an average particle size of about 10 microns, the microcrystalline cellulose particles have an average particle size of about 4 microns, the first portion of boron nitride has an average particle size of about 15 microns, and the second portion of boron nitride has an average particle size of about 47 microns.

20. The method of claim 17, wherein the water is present in an amount of at least 37% by weight of the cosmetic composition.
